(19) United States
(12) Patent Application Publication
(10) Pub. No.: US 2025/0073189 A1
(43) Pub. Date: Mar. 6, 2025

(54) NATURAL DIETARY SUPPLEMENT FOR MENTAL HEALTH

(71) Applicant: Mind Pop LLC, El Segundo, CA (US)

(72) Inventors: Matthew McNamee, El Segundo, CA (US); Kyle Geralds, El Segundo, CA (US); Nicholas Hines, El Segundo, CA (US); William G. Weiland, El Segundo, CA (US)

(73) Assignee: Mind Pop LLC, El Segundo, CA (US)

(21) Appl. No.: 18/239,740

(22) Filed: Aug. 29, 2023

(51) Int. Cl. A61K 31/14; A23L 33/00; A61K 31/522; A61K 31/685

(52) U.S. Cl. CPC A61K 31/14; A23L 33/30; A61K 31/522; A61K 31/685

(57) ABSTRACT

The present invention is a dietary supplement geared toward improvement of mental wellbeing, comprising Alpha-glycerylphosphorylcholine (commonly known as “A-GPC”), Methylliberine, Phosphatidylserine, Caffeine, and Theacrine. It has been found that the foregoing ingredients, when combined, have a synergistic effect that exceeds the contributions of the individual ingredients and provides a robust and effective brain supplement.

### BACKGROUND

Brain supplements, often referred to as nootropics or cognitive enhancers, have a history deeply rooted in ancient civilizations' pursuit of enhancing cognitive abilities. The use of substances like Ginkgo biloba and Bacopa monnieri in traditional Chinese and Ayurvedic medicine, respectively, showcase humanity's long-standing interest in optimizing mental performance. However, it wasn't until the latter half of the 20th century that scientific research and development gained traction in this field. The introduction of compounds like piracetam in the 1960s marked a significant turning point, sparking scientific curiosity into the potential benefits of enhancing cognitive functions through supplementation.

Over the years, research into brain supplements has led to a diverse range of compounds being explored for their potential cognitive-enhancing effects. These supplements often claim to improve memory, focus, creativity, and overall cognitive performance. While some have shown promising results in animal and small-scale human studies, the overall effectiveness of brain supplements remains a topic of debate. The efficacy of these supplements can vary widely based on factors such as individual physiology, dosage, and the specific cognitive domains being targeted.

It's important to note the brain is a complex organ, and cognitive enhancement is a multifaceted endeavor that involves various lifestyle factors. Therefore, a holistic approach that combines healthy habits, mental stimulation, and potential supplementation may yield the most meaningful cognitive benefits over time. While some compounds have shown promise in enhancing cognitive functions, the effectiveness of brain supplements is a nuanced and evolving subject. Ongoing research and advancements in neuroscience may provide a clearer understanding of how these supplements can be used to support cognitive health.

The present invention is a novel brain supplement that is both effective and safe, and the present inventors believe that it provides a unique combination of ingredients that surpasses the effectiveness of the prior art without sacrificing safety and good health.

### SUMMARY OF THE INVENTION

The present invention is the product of many years of testing and evaluation by doctors and scientists. The result is a composition which exhibits a most effective brain health supplement combination. The invention is the unique combination of critical nutrients from a select variety of plants that have both common and uncommon properties, and together, in the ranges set forth herein, yield an extremely unique and effective product that advances brain health in humans.

To wit, a first embodiment of the present invention includes the following ingredients: Alpha-glycerylphosphorylcholine (commonly known as “A-GPC”), Methylliberine, Phosphatidylserine, Caffeine, and Theacrine. It has been found that the foregoing ingredients, when combined, have a synergistic effect that exceeds the contributions of the individual ingredients and provides a robust and effective brain supplement. The relative ratios of the ingredients in a first embodiment is as follows:
Alpha-GPC at 55-60%;
Methylliberine at 10-15%;
Theacrine at 10-15%;
Phosphatidylserine at 9-10%; and
Caffeine 6-10%.

These and other benefits of the present invention will best be understood with reference to the detailed description of the preferred embodiments set forth below.

### DETAILED DESCRIPTION OF THE PREFERRED EMBODIMENTS

In order to create an effective brain supplement it is important to establish a brain to body connection. Through many tests and much research, the present inventors have discovered that alpha-glycerylphosphorylcholine has been shown to increase muscular output and reaction times. It's an advanced form of choline that creates more acetylcholine in the brain, which is the body and brain's primary neurotransmitter. Choline is a recently discovered nutrient—it was only recently acknowledged as a required nutrient by the Institute of Medicine in 1998. Although the human body produces choline in trace quantities, one typically needs to get choline from one's diet to avoid a deficiency. However, many people are not meeting the recommended intake for this nutrient, therefore it is likely to be extremely valuable in supplementation. 600 mg of alpha-glycerylphosphorylcholine, for example, serves a vital function in the present invention's capacity to serve the mind and beyond.

Methylliberine is a little-known alkaloid derived from the kucha tea leaf—its properties include the ability to amplify mood, energy, and focus by activating dopamine receptors and other key neurotransmitters. As a stimulant, it focuses its effects on the central nervous system instead of the cardiovascular system, and thus avoids symptoms known as jitters and/or caffeine crash. That, coupled with the fact that it is structurally similar to caffeine and theacrine, and like those, enhances mood, energy, focus, and motivation in a unique way. Most notably, it has been discovered that in combination with the other ingredients, it produces an unexpected energy boost at precisely 140 mg to potentiate and synergize with caffeine and theacrine. The inventors have discovered that a “+” or “−” of as little as 5 mg significantly affect the results on the brain.

Phosphatidylserine is a type of fat that occurs naturally in every cell in the body—it is found in particularly high concentrations in the brain. In fact, half of the body's total phosphatidylserine resides in the brain. The present inventors found phosphatidylserine to be unexpectedly effective as a supplementary ingredient in assisting proteins that manage membrane functions, assisting the entry of nutrients into cells, effectively modulating cortisol, assisting the exit of waste products out of cells, and transmitting molecular messages from cell to cell. The present invention has been discovered to be particularly effective in cell waste removal. The present invention utilizes, for example, 100 mg to be optimal and work synergistically in the formula, removing the waste in the brain that would otherwise disrupt the total formula's effectiveness.

Caffeine is far and away the most widely known compound in the composition, however, the present invention uses the caffeine as a clean source of energy to go along with methylliberine and theacrine. These three ingredients work together like no others that the present inventors have studied. Methylliberine can deliver a fast acting rush of energy, cognition, mental recall, and vitality to the brain that is followed temporally by energy provided by the caffeine, which amplify these affects, followed by the more slowly released theacrine that prolongs these benefits in a unique and beneficial manner. This combination creates an atypical extended, focused, and relaxed feeling that is amplified to the body by alpha-glycerylphosphorylcholine, and further facilitated by the waste removal of the phosphatidylserine. In addition to the caffeine's crucial interactive and synergistic properties with the aforementioned ingredients, it is important to create and stimulate the growth and differentiation of neurons and synapses in the brain. It thrives in the hippocampus, the cortex, and the basal forebrain which are the areas vital for memory, learning, and higher cognitive functions. At a modest 70 mg of caffeine, the inventors have found tremendous cognitive effects.

Theacrine advances the beneficial effects of the compounds of this invention in unexpected ways. Like caffeine, theacrine stimulates the central nervous system at higher doses and decreases central nervous system activity at lower doses, but unlike caffeine, according to research and trials, theacrine does not seem to significantly affect blood pressure. Throughout the studies pertaining to this invention, it has been discovered that low blood pressure is paramount for the proper firing of synapses and proper chemical balance in the brain. Testing in doses from 50-400 mg of theacrine have narrowed the optimum range to 125-150 mg, and more particularly about 140 mg. At this dosage, theacrine seems to be most effective in calming blood pressure while simultaneously prolonging positive side effects commonly associated with caffeine, therefore showing a unique and unmatched ability to calm and stimulate the mind for prolonged periods of time, which lead to optimal long-lasting brain function. This is made possible by the waste removal from phosphatidylserine and enhanced by alpha-glycerylphosphorylcholine which spreads these effects beyond the brain, throughout the entire body.

The present invention has some of the most effective and unique ingredients that are highly sensitive to the most subtle of adjustments, working powerfully together in a very narrow therapeutic window. This five ingredient formula is an extremely powerful supplement that has the potential to improve the mental health of a large and suffering population.

While the present invention has been referred to in variations of a preferred embodiment, the invention is not limited to any specific embodiment unless expressly so stated. Rather, a person of ordinary skill in the art will readily recognize and appreciate that various substitutions and modifications are possible, and the present invention is intended to include all such substitutions and modifications. Accordingly, the scope of the present invention is properly governed by the words of the appended claims, using their plain and ordinary meanings, consistent with but not limited by the foregoing descriptions.

1. A natural dietary supplement for improving mental health, comprising:
Alpha-Glycerylphosphorylcholine;
Methylliberine in the amount of between 135 and 145 milligrams;
Phosphatidylserine in the amount of 100 milligrams;
Caffeine in the amount of 70 milligrams; and
Theacrine in the amount of 140 milligrams.

2. The natural dietary supplement of claim 1, wherein the Alpha-glycerylphosphorylcholine is in a ratio of fifty five percent to sixty percent (55%-60%) by weight in relation to the Methylliberine, Phosphatidylserine, Caffeine, and Theacrine.

3. The natural dietary supplement of claim 1, wherein the Methylliberine is in a ratio of ten percent to fifteen percent (10%-15%) by weight in relation to the Alpha-glycerylphosphorylcholine, Phosphatidylserine, Caffeine, and Theacrine.

4. The natural dietary supplement of claim 1, wherein the Phosphatidylserine is in a ratio of ten percent to fifteen percent (10%-15%) by weight in relation to the Alpha-glycerylphosphorylcholine, Methylliberine, Caffeine, and Theacrine.

5. The natural dietary supplement of claim 1, wherein the Caffeine is in a ratio of six percent to ten percent (6%-10%) by weight in relation to the Alpha-glycerylphosphorylcholine, Methylliberine, Phosphatidylserine, and Theacrine.

6. The natural dietary supplement of claim 1, wherein the Theacrine is in a ratio of ten percent to fifteen percent (10%-15%) by weight in relation to the Alpha-glycerylphosphorylcholine, Methylliberine, Phosphatidylserine, and Caffeine.

7. A natural dietary supplement, consisting of:
Alpha-Glycerylphosphorylcholine;
Methylliberine;
Phosphatidylserine;
Caffeine; and
Theacrine.
